(19) United States
(12) Patent Application Publication
(10) Pub. No.: US 2005/0101585 A1
(43) Pub. Date: May 12, 2005

(54) Use of selective CB1-antagonists in medical treatments

(75) Inventors: Jochen Antel, Bad Muender (DE); Guenter Krause, Burgdorf (DE); Peter-Colin Gregory, Hannover (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/933,487

(22) Filed: Sep. 3, 2004

Related U.S. Application Data
(60) Provisional application No. 60/524,212, filed on Nov. 24, 2003.

(30) Foreign Application Priority Data
Sep. 3, 2003 (EP) EP 03 10 3284.0

(51) Int. Cl. A61K 31/397; A61K 31/4965; A61K 31/415; A61K 31/34

(52) U.S. Cl. 514/210.01; 514/255.06; 514/406; 514/419; 514/396; 514/471

(57) ABSTRACT

The use of selective CB₁ receptor antagonistic compounds for treating and/or inhibiting CB₁ receptor related diseases in juvenile patients (pediatric treatment), e.g. in particular obesity in juvenile patients, and/or for the treatment and/or inhibition of drug induced obesity in juvenile as well as in adolescent patients, and for the manufacture of pharmaceutical compositions for such purposes.

### CROSS REFERENCE TO RELATED APPLICATIONS

This application claims priority from U.S. provisional patent application No. 60/524,212, filed Nov. 24, 2003, the entire disclosure of which is incorporated herein by reference. Priority is also claimed based on European patent application no. EP 03 10 3284.0, filed Sep. 3, 2003.

### BACKGROUND OF THE INVENTION

The present invention relates to novel therapeutic and/or prophylactic uses of selective CB₁-antagonists and to pharmaceutical compositions containing one or more of these compounds as an active component for the novel uses. The selective CB₁-antagonists addressed in this invention are potent Cannabis-1 (CB₁) receptor antagonists with outstanding utility for the novel medical uses provided by the present invention.

Cannabinoids are present in the Indian hemp Cannabis Sativa L. and have been used as medicinal agents for centuries (Mechoulam, R.; Feigenbaum, J. J. Prog. Med. Chem. 1987, 24, 159). However, only within the past ten years the research in the cannabinoid area has revealed pivotal information on cannabinoid receptors and their (endogenous) agonists and antagonists. The discovery and the subsequent cloning of two different subtypes of Cannabinoid receptors (CB₁ and CB₂) stimulated the search for novel cannabinoid receptor antagonists (Munro, S.; Thomas, K. L.; Abu-Shaar, M. Nature 1993, 365, 61. Matsuda, L. A.; Bonner, T. I. Cannabinoid Receptors, Pertwee, R. G. Ed. 1995, 117, Academic Press, London). In addition, pharmaceutical companies became interested in the development of cannabinoid drugs for the treatment of diseases connected with disorders of the cannabinoid system. The wide distribution of CB₁ receptors in the brain, in combination with the strictly peripheral localization of the CB₂ receptor, makes the CB₁ receptor a very interesting molecular target for CNS-directed drug discovery in the areas of both psychiatric and neurological disorders (Consroe, P. Neurobiology of Disease 1998, 5, 534. Pop, E. Curr. Opin. In CPNS Investigational Drugs 1999, 1, 587. Greenberg, D. A. Drug News Perspect. 1999, 12, 458). Hitherto, three types of distinct CB₁ receptor antagonists are known. Sanofi disclosed their diarylpyrazole congeners as selective CB₁ receptor antagonists. A representative example is SR-141716A, which is currently undergoing Phase II clinical development for psychotic disorders (Dutta, A. K.; Sard, H.; Ryan, W.; Razdan, R. K.; Compton, D. R.; Martin, B. R. Med. Chem. Res. 1994, 5, 54. Lan, R.; Liu, Q.; Fan, P.; Lin, S.; Fernando, S. R.; McCallion, D.; Pertwee, R.; Makriyannis, A. J. Med. Chem. 1999, 42, 769. Nakamura-Palacios, E. M.; Moerschbaecher, J. M.; Barker, L. A. CNS Drug Rev. 1999, 5, 43). Aminoalkylindoles have been disclosed as CB₁ receptor antagonists. A representative example is Iodopravadoline (AM-630), which was introduced in 1995. AM-630 is a CB₁ receptor antagonist, but sometimes behaves as a weak partial agonist (Hosohata, K.; Quock, R. M.; Hosohata, Y.; Burkey, T. H.; Makriyannis, A.; Consroe, P.; Roeske, W. R.; Yamamura, H. I. Life Sc. 1997, 61, PL115). More recently, researchers from Eli Lilly described arylaroyl substituted benzofurans as selective CB₁ receptor antagonists (e.g. LY-320135) (Felder, C. C.; Joyce, K. E.; Briley, E. J.; Glass, M.; Mackie, K. P.; Fahey, K. J.; Cullinan, G. J.; Hunden, D. C.; Johnson, D. W.; Chaney, M. O.; Koppel, G. A.; Brownstein, M. J. Pharmacol. Exp. Ther. 1998, 284, 291). Recently, 3-alkyl-5,5′-diphenylimidazolidinediones were described as cannabinoid receptor ligands, which were indicated to be cannabinoid antagonists (Kanyonyo, M.; Govaerts, S. J.; Hermans, E.; Poupaert, J. H., Lambert, D. M. Biorg. Med. Chem. Lett. 1999, 9, 2233). Interestingly, many CB₁ receptor antagonists have been reported to behave as inverse agonists in vitro (Landsman, R. S.; Burkey, T. H.; Consroe, P.; Roeske, W. R.; Yamamura, H. I. Eur. J. Pharmacol. 1997, 334, R1). Recent reviews provide a nice overview of the current status in the cannabinoid research area (Mechoulam, R.; Hanus, L.; Fride, E. Prog. Med. Chem. 1998, 35, 199. Lambert, D. M. Curr. Med. Chem. 1999, 6, 635. Mechoulam, R.; Fride, E.; Di Marzo, V. Eur. J. Pharmacol. 1998, 359, 1). From the international patent application WO 01/70700 4,5-dihydro-1H-pyrazole compounds are known which exhibit potent and selective cannabis CB₁-receptor antagonistic activity.

### SUMMARY OF THE INVENTION

It is an object of the present invention to provide improved methods of treatment and/or prophylaxis which are particularly suitable in patient groups with enhanced need of safety and tolerability.

Another object of the invention is to provide a method of treatment or inhibition which is particularly suitable for the treatment of obesity patients, especially juvenile obesity patients.

A further object of the invention is to provide a method of treatment and/or prophylaxis for patients subject to long term treatment such as in drug induced obesity, especially in juvenile or adolescent patients.

An additional object of the invention is to provide a method of treating and/or inhibiting CB₁ receptor related diseases, such as psychiatric disorders, neurological disorders, cerebral ischaemia, pain, CNS-diseases involving cannabinoid neurotransmission, gastrointestinal disorders and/or cardiovascular disorders.

These and other objects have been achieved in accordance with the present invention by providing a method of treating or inhibiting a condition selected from the group consisting of CB₁ receptor related diseases and drug induced obesity in a patient in need thereof, said method comprising administering to said patient a pharmaceutically effective amount of a CB₁ receptor antagonistic compound or a prodrug thereof.

It has now surprisingly been found that selective CB₁-antagonists in general, prodrugs thereof, tautomers thereof and salts thereof, show a unique pharmacological profile and therefore are particularly suited for the use in the manufacture of a medicaments for the treatment and/or prophylaxis of obesity patients, in particular of obesity in juvenile patients and/or drug induced obesity in juvenile, as well as adolescent, patients. In this regard selective CB₁-antagonistic compounds are highly valuable in providing medicaments for pediatric use on the one hand, and for the general use in drug induced obesity.

The term “selective” means that preferably there is no substantial other activity than the CB₁-receptor antagonistic activity, or that at least the CB₁-receptor antagonistic activity is substantially overcompensating any other activity.

The outstanding unique pharmacological profile of selective CB₁-antagonistic compounds includes particularly high safety and tolerability which make the compounds particularly suitable in patient groups with enhanced need of safety and tolerability, in particular such as juvenile patients and/or patients subject to long term treatment, e.g. in drug induced obesity.

Due to the potent and selective CB₁ antagonistic activity the compounds used according to the invention are suitable also for use in pediatric treatment and/or prophylaxis of other disorders than juvenile obesity and drug induced obesity in juvenile patients. The other disorders include those known from the literature for the concerned selective CB₁ antagonistic compound, e.g. pediatric treatment and/or prophylaxis may pertain to psychiatric disorders such as psychosis, anxiety, depression, attention deficits, memory disorders and appetite disorders, neurological disorders such as dementia, distonia, Parkinson's disease, Alzheimer's disease, epilepsy, Huntington's disease, Tourette's syndrome, cerebral ischaemia, as well as for the treatment of pain disorders and other CNS-diseases involving cannabinoid neurotransmission, and in the treatment of gastrointestinal disorders and cardiovascular disorders, in young patients.

The entire content of the literature mentioned in the description of the present invention is hereby incorporated by reference into the present application.

The selective CB₁ antagonistic compounds used in the present the invention can be obtained according to known methods. Suitable methods of synthesis for the compounds used according to the present invention are described in the state of the art, e.g. in the documents cited in the present application and incorporated by reference.

Examples of selective CB₁ antagonistic compounds which are useful in the context of the present invention include (without being limited thereto):
1) Diarylpyrazole congeners disclosed by Sanofi as selective CB₁ receptor antagonists, e.g. as representative example the compound SR-141716A, rimonabant and related compounds described e.g. in EP 0969835, SR-147778, SR-140098 (Central mediation of the cannabinoid cue: activity of a selective CB₁ antagonist, SR 141716A Perio A, Rinaldi-Carmona M, Maruani J Behavioural Pharmacology 1996, 7:1 (65-71)); WIN-54461 disclosed by Sanofi-Winthrop (Cannabinoid receptor ligands: Clinical and neuropharmacological considerations relevant to future drug discovery and development. Pertwee R G, Expert Opinion on Investigational Drugs 1996, 5:10 (1245-1253))
2) Aminoalkylindoles having been disclosed as CB₁ receptor antagonists, e.g. as a representative example the compound Iodopravadoline (AM-630),
3) Aryl-aroyl substituted benzofurans described by Eli Lilly as selective CB₁ receptor antagonists, e.g. LY-320135 (Cannabinoid receptor ligands: Clinical and neuropharmacological considerations relevant to future drug discovery and development. Pertwee R G, Expert Opinion on Investigational Drugs 1996, 5:10 (1245-1253)),
4) Compounds described by Merck & Co, e.g. AM 251 and AM 281 (Conference: 31st Annual Meeting of the Society for Neuroscience, San Diego, USA, 10-15.11.2001), and substituted imidazolyl derivatives disclosed e.g. in U.S. 2003-114495 or WO 03/007887,
5) Azetidine derivatives described by Aventis Pharma e.g. in WO 02/28346 or EP 1328269,
6) CP-55940 from Pfizer Inc. (Comparison of the pharmacology and signal transduction of the human cannabinoid CB1 and CB2 receptors, Felder C C, Joyce K E, Briley E M, Mansouri J, Mackie K, Blond O, Lai Y, Ma A L, Mitchell R L, Molecular Pharmacology 1995, 48:3 (443)),
7) Diaryl-pyrazine-amide derivatives from Astra Zeneca described e.g. in the WO 03/051851,
8) ACPA and ACEA from Med. Coll. Wisconsin (Univ. Aberdeen), (“Effects of AM 251 & AM 281, cannabinoid CB1 antagonists, on palatable food intake in lewis rats” J. Pharmacol. Exp. Ther. 289, No 3, 1427-33, 1999),
9) Pyrazole derivatives described by the University of Conneticut e.g. in the WO 01/29007,
10) HU-210 (International Association for the Study of Pain—Ninth World Congress (Part II) Vienna, Austria, Dickenson A H, Carpenter K, Suzuki R, IDDB MEETING REPORT 1999, August 22-27) and HU-243 (Cannabinoid receptor agonists and antagonists, Barth F, Current Opinion in Therapeutic Patents 1998, 8:3 (301-313)) from Yissum R&D Co Hebrew Univ. of Jerusalem,
11) O-823 from Organix Inc. (Drug development pipeline: O-585, O-823, O-689, O-1072, nonamines, Orgaix, Altropane Organix Inc, Company Communication 1999, August 10; IDDb database) and O-2093 from Consiglio Nazionale delle Ricerche (“A structure/activity relationship study on arvanil, endocannabinoid and vanilloid hybrid.”, Marzo DV, Griffin G, Petrocellis L, Brandi I, Bisogno T, Journal of Pharmacology and Experimental Therapeutics 2002, 300:3 (984-991)),
12) 3-Alkyl-5,5′-diphenylimidazolidinediones which were described as cannabinoid receptor ligands,
13) CB₁ antagonistic compounds currently under development by Bayer AG (IDDb database: company communication 2002, Feb. 28).

The CB₁ antagonistic compounds used according to the invention can be brought into forms suitable for pediatric administration, as well as for the administration in treating drug induced obesity by means of usual processes using auxiliary substances and/or liquid or solid carrier materials.

Hence, in a further aspect the invention also pertains to a pharmaceutical composition containing at least one selective CB₁ antagonistic compound as an active component for the treatment and/or prophylaxis of CB₁ receptor related diseases in juvenile patients and/or for the treatment and/or prophylaxis of drug induced obesity in juvenile as well as adolescent patients, and at least one auxiliary excipient. In such a pharmaceutical composition the selective CB₁ antagonistic compound is preferably present in an amount effectively suited for the treatment and/or prophylaxis of a psychiatric disorder, a gastrointestinal disorder, a cardiovascular disorder, or a combination of said disorders, in a juvenile patient in need of such treating.

In a further aspect of the invention, the selective CB₁ antagonistic compound is present in the pharmaceutical composition in an amount effectively suited for the treatment and/or prophylaxis of drug induced obesity in juvenile as well as adolescent patients in need of such treatment.

Finally the invention also includes a method of treatment and/or inhibition of CB₁ receptor related diseases in juvenile patients, in particular juvenile obesity, and/or for the treatment and/or prophylaxis of drug induced obesity in juvenile as well as adolescent patients, characterized in that a compound with selective CB₁ receptor antagonistic activity is administered to said patient in need of such treatment. The method of treatment and/or inhibition according to the invention may be further characterized in that it is a pediatric treatment which is directed to psychiatric disorders such as psychosis, anxiety, depression, attention deficits, memory disorders and appetite disorders, neurological disorders such as Parkinson's disease, dementia, distonia, Alzheimer's disease, epilepsy, Huntington's disease, Tourette's syndrome, ischemia, pain and other CNS-diseases involving cannabinoid neurotransmission, in young patients.

Preferably, in one embodiment of the invention the method of treatment and/or prophylaxis is directed to the treating of obesity in juvenile patients. In another preferred embodiment of the invention the method of treatment and/or prophylaxis is directed to the treating of drug induced obesity in juvenile or adolescent patients. This drug induced obesity may be in particular caused by drugs like atypical antipsychotics.

In one embodiment of the invention the method of treatment and/or prophylaxis is directed to the treating of obesity in juvenile patients. Thus, it is advantageous that Cannabinoid antagonists are suitable for the treatment of Childhood Obesity and related Comorbidities as for example Type 2 Diabetes. There is a clear medical need for improved therapy as obesity has become an increasingly important medical problem not only in the adult population but increasingly in children and (young and older) adolescents. In national surveys from the 1960s to the 1990s in the United States, the prevalence of overweight in children grew from 5% to 11% (Sorof and Daniels 2002). In Canada as another example childhood obesity has tripled in the past 20 years (Spurgeon 2002). Obesity in childhood causes a wide range of serious complications, and increases the risk of premature illness and death later in life, raising public-health concerns (Ebbeling, Pawlak et al. 2002). Over the last decades a tremendous increase of cases of type 2 diabetes was observed, especially also in children. This epidemic trend is clearly reflecting the increasing rates of obesity. Type-2-diabetes was in the past considered a disease of adults and older individuals, not a pediatric condition (Arslanian 2002). One of the main risk factor of pediatric type 2 diabetes is obesity.

Type 2 diabetes in children (as is in adults) is part of the insulin resistance syndrome (Rosenbloom 2002) that includes hypertension, dyslipidemia and other atherosclerosis risk factors, and hyperandrogenism seen as premature adrenarche and polycystic ovary syndrome. Other outcomes related to childhood obesity include left ventricular hypertrophy, nonalcoholic steatohepatitis, obstructive sleep apnea, orthopedic problems, and severe psychosocial problems.

In addition primary hypertension has become increasingly common in children again associated obesity as a major independent risk factor. Obese children are at approximately a 3-fold higher risk for hypertension than non-obese children (Sorof and Daniels 2002). The benefits of weight loss for blood pressure reduction in children have been demonstrated in both observational and interventional studies.

Public concerns are rising because of a rapid development of the childhood obesity epidemic in genetically stable populations. Driving factors are assumed to be mainly adverse environmental factors for which straightforward recommendations of life style modifications exists. Obesity and it's related co-morbidities are very serious medical conditions and state of the art measures and treatment of obesity and especially childhood obesity remain largely ineffective at the time being (Ebbeling, Pawlak et al. 2002). The management of type 2 diabetes in is also especially difficult in children and the adolescent age group (Silink 2002). Craving for and over consumption of palatable food is one of the important factors of life-style related obesity in humans and especially also in children and adolescents. Treatment of type 2 diabetes and other co-morbid conditions by the degree of metabolic derangement and symptoms: The only data on the use of oral hypoglycemic agents in children with type 2 diabetes has been with metformin (Rosenbloom 2002).

Thus, CB₁ antagonists used according to the present invention offer a unique opportunity for the treatment of obesity by interacting with these “driving forces”. They are superior to current medical treatments and especially suited for pediatric treatment because of their outstanding safety profile and/or tolerability. Treatment of obesity especially childhood obesity is besides efficacy dictated by safety.

Obesity in childhood is a medical condition that is likely to require long-term management. The safety profile of CB₁ antagonists according to the present invention are suggested to be superior to current standard medications, and these CB₁ antagonists will be especially suited for the treatment and prevention of childhood obesity and related co-morbidities.

Literature:

Arslanian, S. (2002). “Type 2 diabetes in children: clinical aspects and risk factors.” Horm Res 57 Suppl 1: 19-28.
Ebbeling, C. B., D. B. Pawlak, et al. (2002). “Childhood obesity: public-health crisis, common sense cure.” Lancet 360(9331): 473-82.
Rosenbloom, A. L. (2002). “Increasing incidence of type 2 diabetes in children and adolescents: treatment considerations.” Pediatr Drugs 4(4): 209-21.
Silink, M. (2002). “Childhood diabetes: a global perspective.” Horm Res 57 Suppl 1:1-5.
Sorof, J. and S. Daniels (2002). “Obesity hypertension in children: a problem of epidemic proportions.” Hypertension 40(4): 441-7.
Spurgeon, D. (2002). “Childhood obesity in Canada has tripled in past 20 years.” Bmj 324(7351): 1416.

In another embodiment of the invention the method of treatment and/or prophylaxis is directed to the treatment of drug induced obesity in juvenile or adolescent patients. Drug induced weight gain is also of major concern and subject to high medical need of improved treatments. Again, in this context the CB₁ antagonists according to the present invention are suggested to be superior to current standard medications, and these CB₁ antagonists will be especially suited for the treatment and prevention of drug induced obesity in juvenile as well as in adolescent patients.

Regarding drug induced weight gain, it is reported by Zimmermann, U., T. Kraus, et al. (2003, “Epidemiology, implications and mechanisms underlying drug-induced weight gain in psychiatric patients.” J Psychiatr Res 37(3): 193-220) that body weight gain frequently occurs during drug treatment of psychiatric disorders and is often accompanied by increased appetite or food craving. While occurrence and time course of this side effect are difficult to predict, it ultimately results in obesity and the morbidity associated therewith in a substantial part of patients, often causing them to discontinue treatment even if it is effective. Weight gain appears to be most prominent in patients treated with some of the second generation antipsychotic drugs and with some mood stabilizers. Marked weight gain also frequently occurs during treatment with most tricyclic antidepressants.

Very large weight gains are associated with drugs like for example the atypical antipsychotics clozapine and olanzapine. Some atypical antipsychotics, however, tend to cause significant weight gain, which may lead to poor compliance and other adverse health effects (Nasrallah, H. (2003). “A review of the effect of atypical antipsychotics on weight.” Psychoneuroendocrinology 28 Suppl 1: 83-96.). The mechanisms involved in antipsychotic drug-related weight gain are as yet uncertain, although serotoninergic, histaminic, and adrenergic affinities have been implicated along with other metabolic mechanisms. The atypical antipsychotics vary in their propensity to cause weight change with long-term treatment. Follow-up studies show that the largest weight gains are associated with clozapine and olanzapine, and the smallest with quetiapine and ziprasidone. Risperidone is associated with modest weight changes that are not dose related. Given the equivalent efficacy of atypical antipsychotics, weight-gain profile is a legitimate factor to consider when constructing an algorithm for treatment due to the serious medical consequences of obesity. In this regard co-administration of CB₁ antagonist according to the invention is suggested to work beneficially.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating or inhibiting a condition selected from the group consisting of CB₁ receptor related diseases and drug induced obesity in a patient in need thereof, said method comprising administering to said patient a pharmaceutically effective amount of a CB₁ receptor antagonistic compound or a prodrug thereof.

2. A method according to claim 1, wherein said compound is a tautomeric compound or a pharmaceutically acceptable salt.

3. A method according to claim 1, wherein said condition is a CB₁ receptor related disease, and said patient is a juvenile patient.

4. A method according to claim 1, wherein said condition is drug induced obesity, and said patient is a juvenile or adolescent patient.

5. A method according to claim 1, wherein said compound is a CB₁ antagonistic compound with selective CB₁ receptor antagonistic activity.

6. A compound according to claim 5, wherein said compound is selected from the group consisting of diarylpyrazoles, aminoalkylindoles, aryl-aroyl substituted benzofuran compounds, substituted imidazolyl compounds, azetidine derivatives, diaryl-pyrazine-amide compounds, pyrazole derivatives, and 3-alkyl-5,5′-diphenylimidazolidinediones.

7. A method according to claim 5, wherein said compound is selected from the group consisting of SR-141716A, rimonabant, SR-147778, SR-140098, WIN-54461, Iodopravadoline (AM-630), LY-320135, AM251, AM281, CP-55940, ACPA, ACEA, HU-210, HU-243, O-585, O-823, O-689, O-1072, and O-2093.

8. A method according to claim 1, wherein said condition is obesity in a juvenile patient or drug induced obesity in a juvenile or adolescent patient.

9. A method according to claim 1, wherein said patient is a pediatric patient, and said condition is selected from the group consisting pediatric psychiatric disorders, neurological disorders, cerebral ischaemia, pain disorders, CNS-diseases involving cannabinoid neurotransmission, gastrointestinal disorders, and cardiovascular disorders.

10. A method according to claim 9, wherein said condition is a pediatric psychiatric disorder selected from the group consisting of psychosis, anxiety, depression, attention deficits, memory disorders and appetite disorders, or a neurological disorder selected from the group consisting of dementia, distonia, Parkinson's disease, Alzheimer's disease, epilepsy, Huntington's disease, and Tourette's syndrome.
